(19) United States
(12) Patent Application Publication
(10) Pub. No.: US 2020/0085895 A1
(43) Pub. Date: Mar. 19, 2020

(54) PUNICALAGIN COMPOSITIONS AND METHODS FOR THE TREATMENT OF DISORDERS ARISING FROM METHYLGLYOXAL-INDUCED DNA DAMAGE

(71) Applicant: Verdure Sciences, Noblesville, IN (US)

(72) Inventors: Ajay Patel, Noblesville, IN (US); Sonya Cropper, Noblesville, IN (US)

(21) Appl. No.: 16/570,483

(22) Filed: Sep. 13, 2019

Related U.S. Application Data
(60) Provisional application No. 62/730,599, filed on Sep. 13, 2018.

(51) Int. Cl. A61K 36/185; A61K 31/352

(52) U.S. Cl. CPC A61K 36/185; A61K 31/352

(57) ABSTRACT

The present invention provides in methods for reducing or inhibiting DNA damage in a cell. The methods administer compositions of at least a pomegranate polyphenol to reduce or inhibit DNA damage in the cell.

### CROSS-REFERENCE TO RELATED APPLICATIONS

This application claims the benefit of U.S. Provisional Application No. 62/730,599, filed on Sep. 13, 2018, the contents of which are incorporated by reference in their entirety.

### FIELD OF THE INVENTION

DNA damage plays a role in a myriad of diseases, especially age related diseases. The present disclosure relates to compositions and methods of inhibiting or reducing DNA damage and diseases associated thereof.

### BACKGROUND OF THE INVENTION

Skin damage, such as sunburn, results in an acute inflammatory response in the skin tissue due to excessive ultraviolet radiation or environmental toxins. Tissue damage results, for example, from direct radiation-induced damage or damage caused by oxygen free radicals.

Direct radiation-induced damage in skin tissue results from absorption of high-energy ultraviolet radiation by biomolecules. Ultraviolet radiation type B having a wavelength between 280 and 320 nm is absorbed by DNA, resulting in the formation of products such as cyclobutane pyrimidine dimers (CPD) and pyrimidine-6,4-Pyrimidion (6,4PP) by covalent bonding. These products induce double strand breaks and inhibit DNA repair mechanism.

Ultraviolet radiation can induce the formation of reactive oxygen species (ROS) in exposed tissue. Another type of oxidative stress stems from methylglyoxal, an endogenous metabolic by-product of glycolysis. The resulting oxidative stress leads to immediate oxidative DNA damage, which inhibits the cellular DNA repair mechanism.

Recent research has shown that pomegranate (Punica granatum L.) juice and pomegranate extracts exhibit potent biological properties attributable to the presence of polyphenols known as ellagitannins or punicalagins. These hydrolysable tannins or calagins are present in high levels in pomegranates and have been identified as the active antioxidant compounds responsible for protecting low-density lipoprotein cholesterol from oxidation in vivo, a key step in the pathogenesis of atherosclerosis.

Although the punicalagins antioxidant effect has been studied in cholesterol disease, little is known about the antioxidant effect of punicalagins in DNA damage. This disclosure proposes compositions and methods of inhibiting or reducing DNA damage and diseases associated thereof by using punicalagins.

### BRIEF DESCRIPTION OF THE DRAWINGS

FIG. 1 demonstrates that punicalagin has a protective effect against methylglyoxal (MGO)-induced DNA damage.

FIG. 2 is a schematic representation of (A) formation of urolithin A via microbial metabolism (B) formation of damaged DNA by methylglyoxal (MGO).

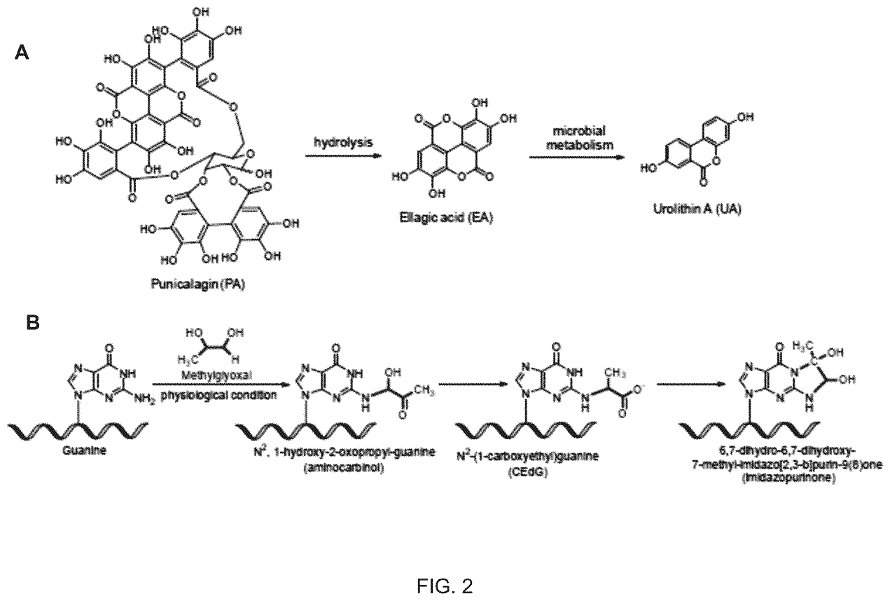

FIG. 3 demonstrates the effects of different concentration of (A) PE, (B) PA, (C) EA and (D) UA on viability of HaCaT cells.

FIG. 4 demonstrates effects of MGO on viability of HaCaT cells. (A) HaCaT cells were treated with MGO at concentrations of 100, 200, 300, 400, 500 and 600 μM. Cell viability was measured by using CTG 2.0 assay and (B) are representative images of HaCaT cells exposed to MGO for 24 h and then stained with crystal violet. Results demonstrate the addition of increasing amount of MGO to HaCaT cells results in increased cell death.

FIG. 5 shows the protective effects of PE and pomegranate phenolics on cell viability against MGO induced glycative stress. (A) Effects of PE (top-left graph), PA (top-right graph), EA (bottom-left graph), and UA (bottom-right graph) on cell viability of HaCaT cells exposed to MGO (400 μM). Cell viability was measured by using CTG 2.0 assay. (B) Representative images of HaCaT cells pre-treated with PE (50 μg/mL), PA (50 μM), EA (50 μM), and UA (50 μM) of the compounds for 2 hours followed by wash with PBS twice and treatment with 400 μM MGO for 24 hours. The morphological changes of HaCaT cells were observed by an EVOS Cell Imaging System (Invitrogen, Waltham, Mass., USA) in 6-well plates after crystal violet stain.

FIG. 6 demonstrates the protective effects of PE and pomegranate phenolics against MGO induced reactive-oxygen species (ROS). (A) graphs showing the cellular ROS level after 24 h when cells are treated with MGO and PE (top-left graph), MGO and PA (top-right graph), MGO and EA (bottom-left graph), and MGO and UA (bottom-right graph). (B) shows the representative figures of the data demonstrated in (A).

FIG. 7 demonstrates the protective effect of HaCaT cell on DNA. Immunofluorescence representative pictures of HaCaT cells treated with PE, PA, EA, and UA. HaCaT cells were stained with Hoechst 33342. (A) control, (B) MGO 400 μM, (C) PE 50 μg/ml+MGO 400 μM, (D) PA 50 μM+MGO 400 μM, (E) EA 50 μM+MGO 400 μM, and (F) UA 50 μM+MGO 400 μM. (original magnification ×10).

FIG. 8 demonstrates the protective effect of PE and pomegranate phenolics on DNA integrity of HaCaT cells by reducing MGO-induced DNA damage. Immunofluorescent images of SYBR GOLD stained comet assay image (A) control, (B) MGO 400 μM, (C) PE 50 μg/ml+MGO 400 μM, (D) PA 50 μM+MGO 400 μM, (E) EA 50 μM+MGO 400 μM, and (F) UA 50 μM+MGO 400 μM. (G) Tail DNA percentage of random 50 cells in each group analyzed by CASP (original magnification ×10, scale 400 μm).

FIG. 9 demonstrates the protective effect of PE and pomegranate phenolics on HaCaT cell in cell adhesion and migration. (A) is a graph demonstrating the cell adhesion rate % when cells are treated with MGO alone and in combination with PE, PA, EA, and UA, respectively. (B) is a graph demonstrating the cell migration ability when cells are treated with MGO in combination with PE, PA, EA, and UA, respectively. (C) are images demonstrating xxx CON are cells non-treated with MGO, MGO indicates cells treated with MGO, PE MGO indicates cells treated with MGO and PE, PA MGO indicates cells treated with MGO and PA, EA MGO indicates cells treated with MGO and EA, and UA MGO indicates cells treated with MGO and UA. (D) is a graph demonstrating the healing rate % when cells are treated with MGO alone and in combination with PE, PA, EA, and UA, respectively. (E) are images demonstrating cell migration where CON are cells non-treated with MGO, MGO indicates cells treated with MGO, PE MGO indicates cells treated with MGO and PE, PA MGO indicates cells treated with MGO and PA, EA MGO indicates cells treated with MGO and EA, and UA MGO indicates cells treated with MGO and UA, images are captures at 0 h and 48 h after treatment.

### SUMMARY OF THE INVENTION

The present invention provides a method of reducing or inhibiting DNA damage in a cell, the method comprising administering an effective amount of a pomegranate polyphenol composition comprising at least a pomegranate polyphenol to reduce or inhibit DNA damage in the cell.

In an embodiment, the pomegranate polyphenol composition comprises a punicalagin-enriched extract from pomegranate.

In an embodiment, the pomegranate polyphenol composition comprises punicalagin, ellagic acid, urolithin A, urolithin B, urolithin C, urolithin D, urolithin E, urolithin M, gallic acid, pomegranate extract, or a combination thereof.

In an embodiment, the pomegranate polyphenol composition comprises a combination of punicalagin and ellagic acid, wherein the combination of punicalagin and ellagic acid is from 25% to 40% punicalagin and from 2.0 to 3.0% ellagic acid by weight.

In an embodiment, the pomegranate polyphenol composition composition comprises a combination of punicalagins, ellagic acid, and urolithins, wherein the combination of punicalagins, ellagic acid, and urolithins is from 10% to 50% PA, from 0.5% to 5% EA, and from 0.5% to 20% urolithins by weight.

In some embodiments, the present invention provides a method of reducing or preventing MGO binding, and/or MGO induced protein glycation in a subject in need thereof, the method comprising administering an effective amount of a pomegranate polyphenol composition comprising one or more pomegranate polyphenol to reduce or prevent MGO binding and/or MGO induced protein glycation in at least one cell of the subject.

In further embodiments, the present invention provides a method of treating a skin damage disease caused by DNA damage, the method comprising administering an effective amount of a pomegranate polyphenol composition comprising at least a pomegranate polyphenol to reduce or inhibit the skin damage.

### DETAILED DESCRIPTION OF THE INVENTION

The present disclosure demonstrates that pomegranate extract comprising punicalagins has a protective effect against MGO-induced DNA damage both when used topically and orally.

The present invention provides in one embodiment a method for reducing or inhibiting DNA damage in a cell, the method comprising administering an effective amount of a composition comprising at least a pomegranate polyphenol to reduce or inhibit DNA damage in the cell. In a preferred embodiment, the pomegranate polyphenol is pomegranate extract. In an embodiment, the pomegranate extract comprises a combination of punicalagin and ellagic acid. In another embodiment, the pomegranate extract comprises a combination of punicalagins and urolithins. In an embodiment, the pomegranate extract comprises a combination of punicalagins and urolithin A. In an embodiment, the composition comprises punicalagin-enriched extract from a pomegranate. In an embodiment, the pomegranate extract comprises punicalagin, gallic acid, ellagic acid, or a combination thereof.

Urolithins are metabolite compounds that result from the transformation of punicalagins (also known as ellagitannins). Non limiting examples of urolithins are urolithin A (UA), urolithin B (UB), urolithin C (UC), urolithin D (UD), urolithin E (UE), urolithin M (UM), and the like.

In an embodiment, the pomegranate polyphenol composition includes punicalagins (PA), ellagic acid (EA), urolithins, or a combination thereof. In an embodiment, the polyphenol composition comprises a combination of PA and EA. In an embodiment, the combination of PA and EA is from 3% to 95% by weight. In an embodiment, the combination of PA and EA is from 10% to 90% PA and up to 10% EA by weight. In an embodiment, the combination of PA and EA is from 10% to 50% PA and from 0.5% to 5% EA by weight. In an embodiment, the combination of PA and EA is from 25% to 40% PA and from 2.0% to 3.0% EA by weight.

In an embodiment, the polyphenol composition comprises a combination of PA, EA, and urolithins. In an embodiment, the combination of PA, EA, and urolithins is from 3% to 95% by weight. In an embodiment, the combination of PA, EA, and urolithins is from 10% to 90% PA, up to 10% EA, and from 0.5% to 55% urolithins by weight. In an embodiment, the combination of PA, EA, and urolithins is from 10% to 50% PA, from 0.5% to 5% EA, and from 0.5% to 20% urolithins by weight. In an embodiment, the combination of PA, EA, and urolithins is from 25% to 40% PA, from 2.0% to 3.0% EA, and from 5.0% to 10% urolithins by weight.

Suitable methods of making pomegranate extracts, including punicalagin-enriched extracts from pomegranates are described in U.S. Pat. No. 7,897,791, the contents of which are incorporated by reference in its entirety.

Included in the compositions used in the methods are pomegranate polyphenols or pomegranate extracts comprising punicalagins, which may be present as a component of the total pomegranate tannins or may be isolated from other tannins present in pomegranate polyphenols or pomegranate extracts. Punicalagins may be present at a concentration of at least about 5%, at least about 10%, or more.

The compositions of the invention may be provided as a composition containing a pharmaceutically acceptable carrier. Such dosage forms encompass physiologically acceptable carriers that are inherently non-toxic and non-therapeutic. Examples of such carriers include ion exchangers, soft gels, oils, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of tannins include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The extracts will typically be formulated in such vehicles at a concentration of about 0.1 μg/ml to 100 μg/ml and higher. Nutraceutical formulations of interest include foods for veterinary or human use, including health food bars, drinks and drink supplements, and the like. These foods are enhanced by the inclusion of a biologically active extract of the invention for the methods of used described herein.

For cosmetic formulations, the compositions of the invention may optionally comprise skin benefit materials. These include estradiol, progesterone, pregnanalone, coenzyme Q10, methylsolanomethane (MSM), copper peptide (copper extract), plankton extract (phytosome), glycolic acid, kojic acid, ascorbyl palmitate, all-trans-retinol, azaleic acid, salicylic acid, broparoestrol, estrone, adrostenedione, androstanediols, etc. The steroids will generally be present at a concentration of less than about 2% of the total by weight of the composition, while the other skin benefit materials may be present at higher levels, for example as much as 10 to 15%.

The compositions of the invention may comprise a cosmetically acceptable vehicle to act as a diluent, dispersant or carrier, so as to facilitate its distribution when the composition is applied to the skin. Vehicles other than, or in addition to, water can include liquid or solid emollients, solvents, humectants, thickeners and powders.

The cosmetically acceptable vehicle will usually form from 0.1%, or 5% to 99.9%, preferably from 25% to 80% by weight of the composition, and can, in the absence of other cosmetic adjuncts, form the balance of the composition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, intraaural administration, rectal administration, sublingual administration, buccal administration, and parenteral administration, including injectable such as intravenous administration, intra-arterial administration, intramuscular administration, intradermal administration, intrathecal administration and subcutaneous administration. Administration can be continuous or intermittent. In various aspects, a preparation can be administered therapeutically, that is, administered to treat an existing disease or condition. Routes of administration are preferably orally or topically.

For purposes of the present invention, “treating” or “treatment” describes the management and care of a subject for the purpose of combating the disease, condition, or disorder. Treating includes the administration of a composition of the present invention to prevent the onset of the symptoms or complications, alleviating the symptoms or complications, or eliminating the disease, condition, or disorder.

In an embodiment, the present compositions may be used to treat a skin disorder, for example skin damage due to DNA damage. Skin damage may be caused by an underlying disease, environmental toxins or UVA/UVA exposure (i.e., sunburn). The present invention provides method of treating skin damage comprising administering an effective amount of a pomegranate polyphenol composition comprising at least one pomegranate polyphenol to reduce or inhibit skin damage. Skin damage can also include collagen breakdown within the skin. The compositions of the present invention are able to provide skin protection from DNA damage to epithelial cells.

In an embodiment, the present composition is a cosmetic composition comprising at least one pomegranate polyphenol to be used topically to aid in improvement of skin and to reduce or inhibit skin damage.

In another embodiment, the present compositions may be used for treating age-related disorders. In one embodiment, the present invention provides a method of reducing one or more symptoms associated with age-related disorders, the method comprising administering an effective amount of a pomegranate polyphenol composition comprising at least one pomegranate polyphenol to reduce or inhibit at least one symptom of the age related disorder.

As used herein, the terms “effective amount” and “therapeutically effective amount” refer to the quantity of active therapeutic agent or agents sufficient to yield a desired therapeutic response without undue adverse side effects such as toxicity, irritation, or allergic response. The specific “effective amount” will, obviously, vary with such factors as the particular condition being treated, the physical condition of the subject, the type of animal being treated, the duration of the treatment, the nature of concurrent therapy (if any), and the specific formulations employed and the structure of the compounds or its derivatives.

In some embodiments, the DNA damage is from MGO binding, and/or MGO induced protein glycation. DNA damage due to other factors are also contemplated to be able to be reduced by the compositions and methods of the present invention.

The disclosure also provides a method of preventing or reducing MGO binding, and/or MGO induced protein glycation in a subject in need thereof, the method comprising administering an effective amount of a composition comprising one or more polyphenols to prevent or reduce MGO binding and/or MGO induced protein glycation in cells of the subject.

In some embodiments, the cell is treated in vivo.

In some embodiments, the cells treated are skin cells, or neural cells.

In some embodiments, the cells treated are cells normally damaged when a subject suffers from diabetes or a diabetic condition.

As used herein “subject” or “patient” refers to mammals and non-mammals. “Mammals” means any member of the class Mammalia including, but not limited to, humans, non-human primates such as chimpanzees and other apes and monkey species, farm animals such as cattle, horses, sheep, goats, and swine, domestic animals such as rabbits, dogs, and cats, laboratory animals including rodents, such as rats, mice, and guinea pigs, and the like. Examples of non-mammals include, but are not limited to, birds, and the like. The term “subject” does not denote a particular age or sex. In one specific embodiment, a subject is a mammal, preferably a human.

This compositions and methods of the invention may be used for the treatment of age related disorders, specifically in the ability to prevent or reduce MGO binding, and/or MGO induced protein glycation. Further, the compositions and methods may be used for preventing or reducing the formation of advanced glycation end-products and/or protective effects against glycation.

Some areas of health in which the methods described herein may be used include, but are not limited to, for example, metabolic, anti-aging, brain health, skin protective, gut/digestive, inflammation, autoimmune disorders, collagen-breakdown from soft tissue of skin, neurodegenerative diseases, among others.

Methods of preparing compositions comprising polyphenols and/or punicalagrin are described in U.S. Pat. Nos. 7,638,640, 7,897,791, and 7,919,636, the contents of which are incorporated by reference in their entireties.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim.

The invention will be more fully understood upon consideration of the following non-limiting examples.

### EXAMPLES

### Example 1: Punicalagins can Protect Against DNA Damage

Materials

MGO, crystal violet powder (C0775) and 10><trypsin solutions were purchased from Sigma-Aldrich (St. Louis, Mo., US). Dulbecco's modified Eagle's medium (DMEM) and fetal bovine serum (FBS) were supplied by Gibico BRL (Ground Island, N.Y., US). CellTiter-Glo 2.0 Cell Viability Assay was purchased from Promega (Fitchburg, Wis., US). Dimethyl sulfoxide (DMSO) and 2′,7′-dichlorouorescin diacetate (DCFDA) were purchased from Sigma Chemical Co. (St. Louis, Mo., USA). Hoechst (33342) and Alexa Fluor® 488 Annexin V/Dead Cell Apoptosis Kit was brought from Thermo Fisher Scientific (Waltham, Mass., US). CometAssay was purchased from TREVIGEN (Gaithersburg, Md., USA). Pomegranate extract (PE), punicalagin (PA), urolithin A(UA), and ellagic acid (EA) were extracted and purified as previously reported.¹

Experimental Methods and Results

DNA Oligonucleotide Synthesis. Nine-mer single stranded DNA oligonucleotide (9mer ss-DNA) with the sequence 5′-TTTTGTTTT-3′ containing a single guanine base was synthesized by using solid-phase phosphoramidite chemistry as previously described² on MerMade-4 DNA synthesizer. The oligonucleotide was purified by using Thermo Scientific DNAPac PA100 Oligonucleotide anion-exchange column (4×250 mm, 13 μm) with two mobile phases. Solvent A was water, and solvent B was 1.5M ammonium acetate in water. The oligonucleotide was characterized by HPLC electrospray ionization (ESI) triple quadrupole-TOF mass spectrometry (MS). The concentration of oligonucleotide was determined by measuring UV absorbance at 260 nm on a NanoDrop spectrophotometer.

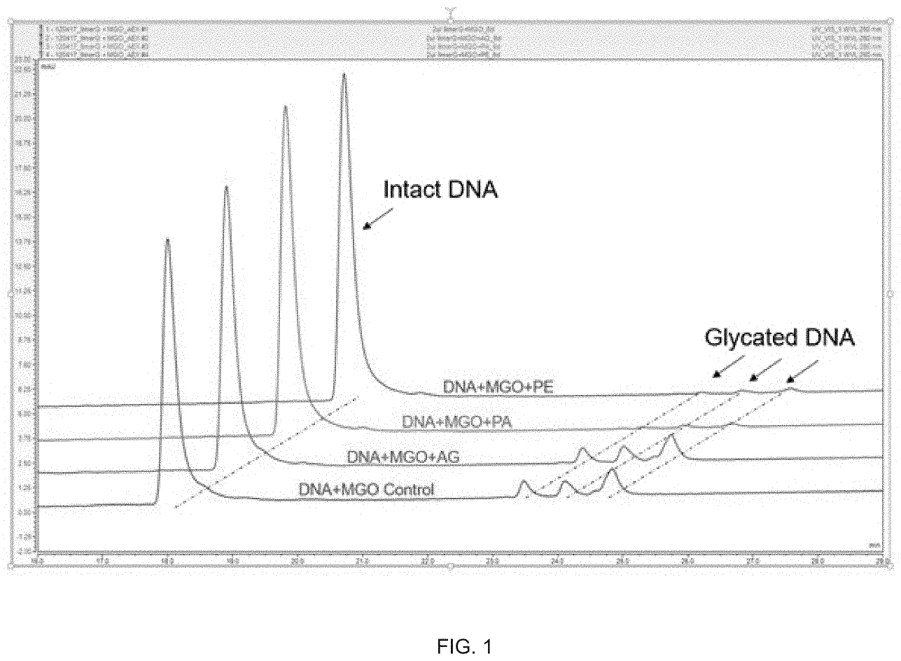

Glycation Assay and LC-MS Analysis. As shown in FIG. 1, cells were treated MGO. All glycation reactions either untreated or treated with natural products (AG 100 μM, PA 100 μM, and PE 100 μM, respectively) were incubated under physiological conditions (pH 7.4, 37° C.) in PBS buffer containing 200 μM 9mer ss-DNA and 200 μM MGO for 7 days. LC-MS analysis was carried out on AB Sciex triple quadrupole-TOF 4600 mass spectrometer. ESI was conducted under a negative ion mode by applying the following parameters: needle voltage at 4.5 kV, heater temperature at 300° C., nebulizer gas at 30 psi, heater gas at 40 psi, curtain gas at 30 psi, declustering potential at −100V and the collision energy at −10 V. Liquid chromatographic separation was performed by using Thermo Scientific Accucore 150 Amide HILIC Column (3×100 mm; 2.6 μm) at a flow rate of 0.3 mL/min. The solvent A was 15 mM ammonium acetate in 90% acetonitrile, solvent B was 15 mM ammonium acetate in 10% acetonitrile. A linear gradient was used under the following conditions: 30% of B for 5 min, 30-60% of B over 15 min, 60% of B over 5 min, 60-30% of B for 1 min and 30% of B over 15 min. Data analysis was performed with AB Sciex Analyst TF software 1.7.

Statistical Analysis. Statistical analysis was conducted by IBM SPSS Statistics 23 and Microsoft Excel 2016. The data between two groups was assessed under the two-tailed Student's t test. p-value<0.05 was considered as statistically significant.

Samples were treated with punicalagins or aminoguanidine (AG) as a positive control. As shown in FIG. 1, Punicalagins were considerably more active at an equivalent concentration (50 μM) to reducing DNA damage.

### Example 2: Cell Viability Studies

Materials and Methods.

Cell Culture.

HaCaT cells which were derived from human adult keratinocytes were bought from American Type Culture Collection (Manassas, Va., USA). The cells were maintained in DMEM supplemented with 5% FBS at 37° C. under an atmosphere of 5% CO2 and 95% air. The culture medium was replaced every other day.

Cell Viability and Viability of Different Experimental Compounds on HaCaT Cells.

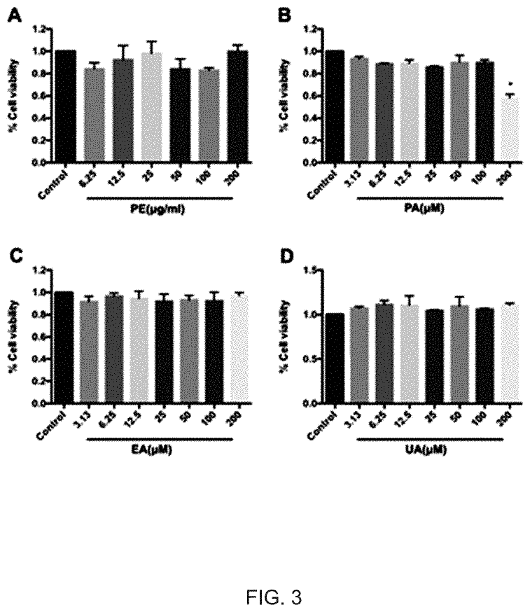

Cellular viability was assessed using the Cell Titer Glo2.0 (CTG 2.0) one-step assay. Briefly, HaCaT cells were seeded at about 50 000 cells/ml to yield 50-60% confluence in a standard white-walled clear bottom 96-well plate. HaCaT cells were exposed to different concentration of PE (0, 6.25, 12.5, 25, 50, 100, 200 μg/mL), PA (0, 3.13, 6.25, 12.5, 25, 50, 100, 200 μM), EA (0, 3.13, 6.25, 12.5, 25, 50, 100, 200 μM), and UA (0, 3.13, 6.25, 12.5, 25, 50, 100, 200 μM) for 24 hours. Following the pre-determined incubation period of 24 hours, CTG 2.0 was added in a 1:1 ratio with existing media and mixed for 5 minutes on an orbital shaker prior to luminescence measurement (Spectramax M2, Molecular Devices, Sunnyvale, Calif., USA). As shown in FIG. 3, PE, EA and UA are not cytotoxic to HaCaT cells in the concentration range tested.

MGO Induced Cell Damage Model.

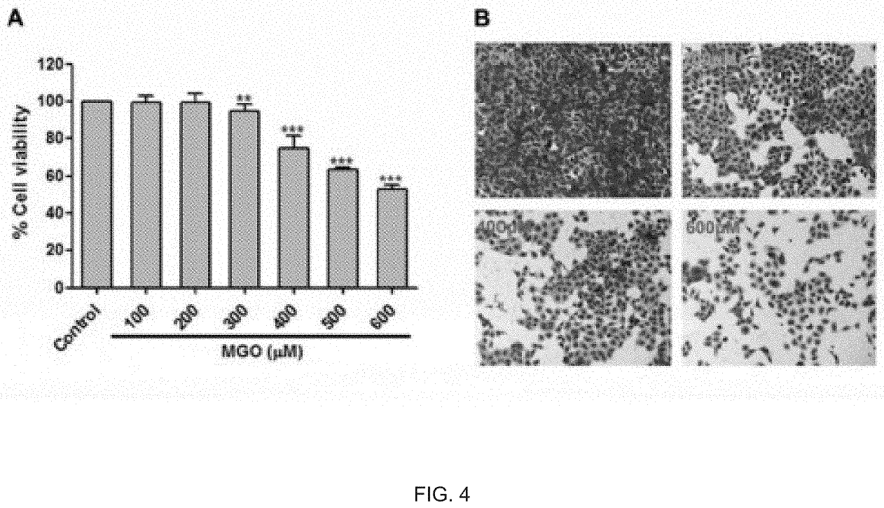

Briefly, HaCaT cells were seeded at about 50000 cells/ml to yield 50-60% confluence in standard white-walled clear bottom 96-well plates and 6-well plates. After 12 h incubation cells were treated with different concentrations of MGO (10004, 200 μM, 300 μM, 400 μM, 500 μM and 600 μM) for 24 hours respectively and use non-treated group as control. Cellular viability was detected using cells in 96-well plates via CTG 2.0. As shown in FIG. 4, the preferred concentration of 400 μM MGO was used in further studies, to evaluate the potential protection properties of testing extract or compounds. MGO concentrations from 100-300 μM did not affect the cell viability. At higher concentrations than 400 μM the damaged caused by MGO to the cells was extreme. Morphological analysis was conducted to evaluate cell damage with crystal violet staining method in 6-well plates as per the below mentioned procedure.

Crystal Violet Staining.

Briefly crystal violet staining were carried with protocol provided by sigma. The staining solution was prepared by dissolve 5 g of crystal violet powder into 100 ml 20% ethanol to make. HaCaT cells in 6-well plates were fixed with 75% ethanol 15 minutes in room temperature. Then cells were incubated with staining solution for 10 minutes. After that cells were washed with PBS for 5 times. Subsequently, pictures were taken under EVOS Cell Imaging System (Invitrogen, Waltham, Mass., USA).

Results and Discussion

Excessive accumulation of MGO is harmful for skin cells, so the effects of MGO on cell viability at various concentrations on HaCaT cells were evaluated first. The results from the cell viability and staining assays showed that at lower concentrations (100 to 300 μM MGO), treatment of MGO was non-toxic; however, MGO at 400 μM or higher concentrations caused significant cytotoxicity. Since MGO at a concentration higher that 400 μM was found extremely cytotoxic, a concentration of 400 μM MGO was used as an inducer of glycative stress in HaCaT cells for further experiments.

Protective Effect of Compounds on MGO Induced Cell Damage Model

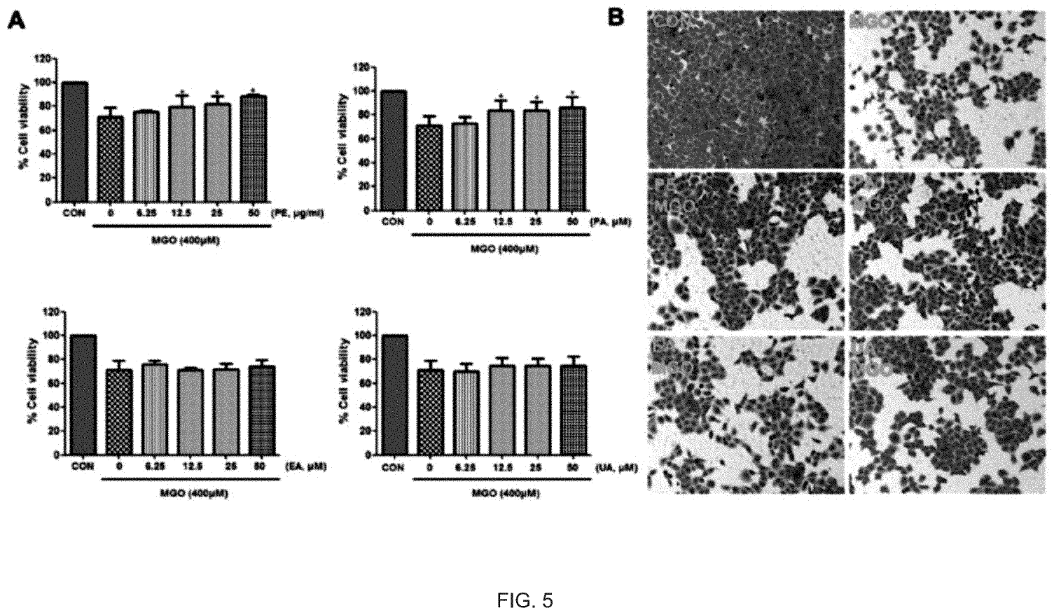

Briefly, HaCaT cells were seeded at about 50000 cells/ml to yield 50-60% confluence in standard white-walled clear bottom 96-well plates and 6-well plates. After 12 h incubation cells were pre-treated with different concentrations of PE (0, 6.25, 12.5, 25, 50, 100, 200 μg/mL), PA (0, 3.13, 6.25, 12.5, 25, 50, 100, 200 μM), EA (0, 3.13, 6.25, 12.5, 25, 50, 100, 200 μM), and UA (0, 3.13, 6.25, 12.5, 25, 50, 100, 200 μM) for 2 hours. Then cells were washed with PBS twice and treated with 400 μM MGO for 24 hours. Cellular viability was detected using cells in 96-well plate via CTG 2.0, and cell images were taken with EVOS Cell Imaging System (Invitrogen, Waltham, Mass., USA) using cells in 6-well plates after crystal violet stain. As shown in FIG. 5, treatment of PE (12.5, 25, and 50 μg/mL) and PA (12.5, 25, and 50 μM) significantly increased cell viability in HaCaT cells compared to the non-treated group. Non-significant change in the cell viability compared to non-treated group was observed at the test concentrations.

Reactive Oxygen Species (ROS) Assay

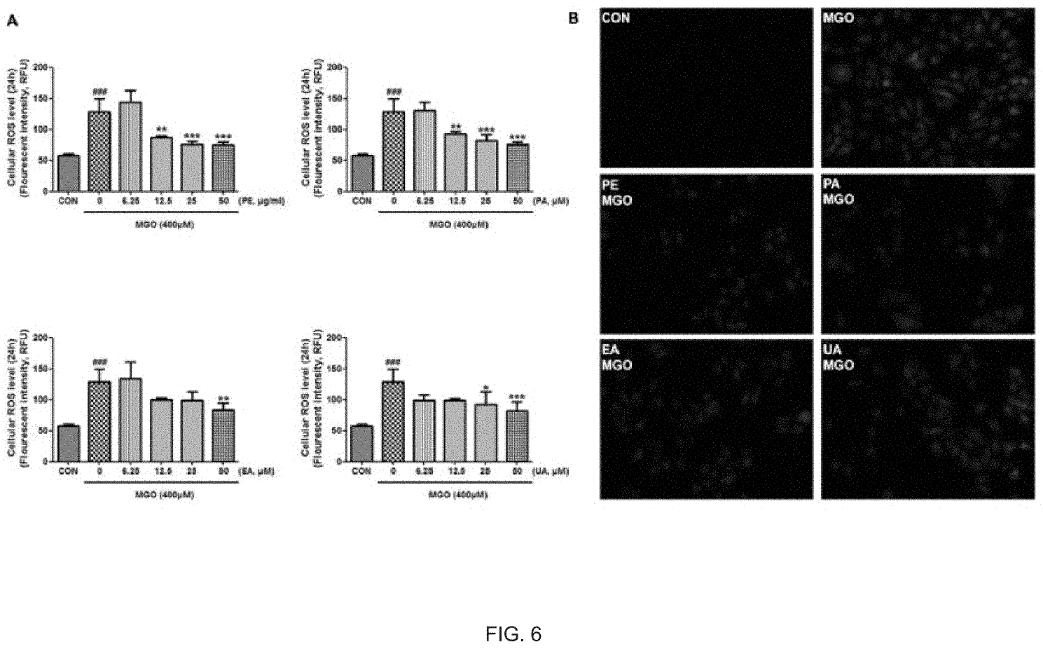

HaCaT cells were seeded at about 50 000 cells/ml to yield 50-60% confluence in standard white-walled clear bottom 96-well plates and 6-well plates in DMEM. After 12 h incubation cells were pre-treated with concentrations of PE (0, 6.25, 12.5, 25, 50, 100, 200 μg/mL), PA (0, 3.13, 6.25, 12.5, 25, 50, 100, 200 μM), EA (0, 3.13, 6.25, 12.5, 25, 50, 100, 200 μM), and UA (0, 3.13, 6.25, 12.5, 25, 50, 100, 200 μM) for 2 hours. Then, the medium was replaced with DMEM containing 20 μM DCFDA and MGO (400 μM) for 24 h. After incubation, all the wells were washed with PBS twice. Subsequently, the fluorescence signals from 96-well plates were read at excitation and emission wavelengths of 485 and 525 nm, respectively, using a Spectra Max M2 spectrometer (Molecular Devices, Sunnyvale, Calif., USA). Cells images were taken with EVOS Cell Imaging System (Invitrogen, Waltham, Mass., USA) using 6-well plates cells. As shown in FIG. 6, treatment with PE (12.5, 25, and 50 μg/mL), PA (12.5, 25, and 50 μM), EA (50 μM) and UA (25 and 50 μM) significantly reduced the production of MGO induced ROS in HaCaT cells as compared to non-treated group.

Protective Effects on HaCaT Cell DNA

Hoechst Staining

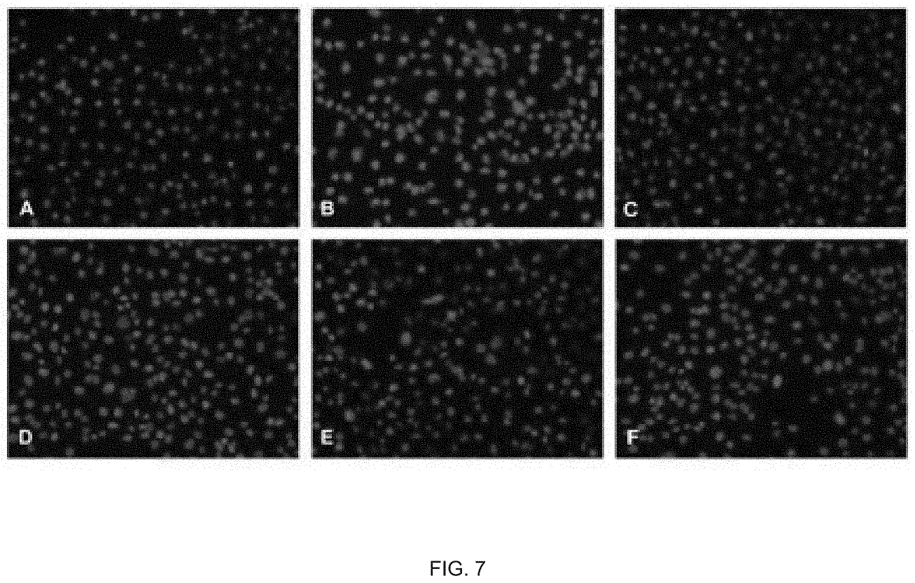

HaCaT cells were seeded at about 50,000 cells/ml to yield 50-60% confluency in a standard 6-well plate. Cells were pre-treated with PE 50 μg/mL, PA 50 μM, EA 50 μM, and UA 50 μM, respectively for 2 hours. Then cells were washed with PBS once and treated with 400 μM of MGO for 24 hours. After that cells were washed with PBS twice and then incubated with 250 μl Hoechst 33342 (1:300 dilute with PBS) at room temperature for 15 minutes. Then cells were fixed with 75% ethanol for 15 minutes, followed by imaging with EVOS Cell Imaging System (Invitrogen, Waltham, Mass., USA). As shown in FIG. 7, PE, PA, EA and UA can reduce the DNA damage level caused by MGO.

Comet Assay

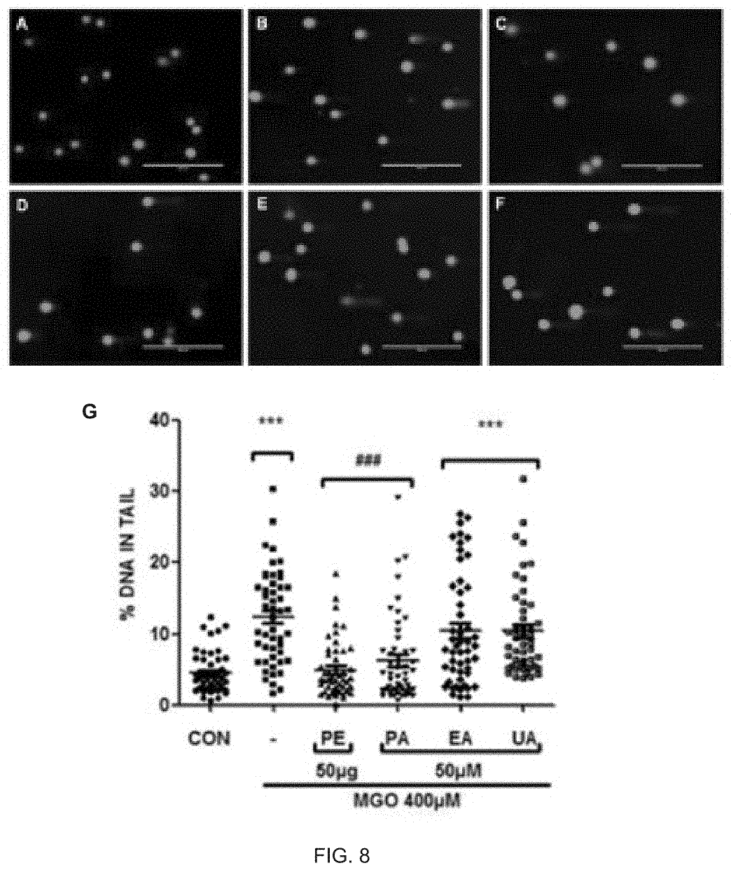

HaCaT cells were seeded at about 100 000 cells/ml to yield 80-90% confluence in standard 6-well plates. Then cells were pre-treated with PE 50 μg/mL, PA 50 μM, EA 50 μM, and UA 50 μM, respectively for 2 hours. After incubation, the cells were washed with PBS twice and then treated with MGO (400 μM) for 24 h. After that, comet assay was performed as per the protocol provided by TREVIGEN (4250-050-K). Briefly, cells were collected and combined with molten LMAgarose at a ratio of 1:10, pipetted onto Comet slides, incubate in Lysis Solution at 4° C. overnight, immerse slides in Alkaline Unwinding Solution for 20 minutes, apply electrophoresis in Alkaline Electrophoresis Solution at 21 volts for 30 minutes, fixed in 75% ethanol for 15 minutes, dry in 37° C. for 15 minutes and then stain in diluted SYBR GOLD, images were taken with EVOS Cell Imaging System (Invitrogen, Waltham, Mass., USA) and analyzed via CASP. An alkaline comet assay was performed to evaluate the DNA damage induced by MGO and it showed MGO could increase the percentage of fracture DNA compared to control group. As shown in FIG. 8, comet assay showed the protective effect of PE and PA on MGO induced DNA. While EA and UA have no protective effects.

Adhesion Assay

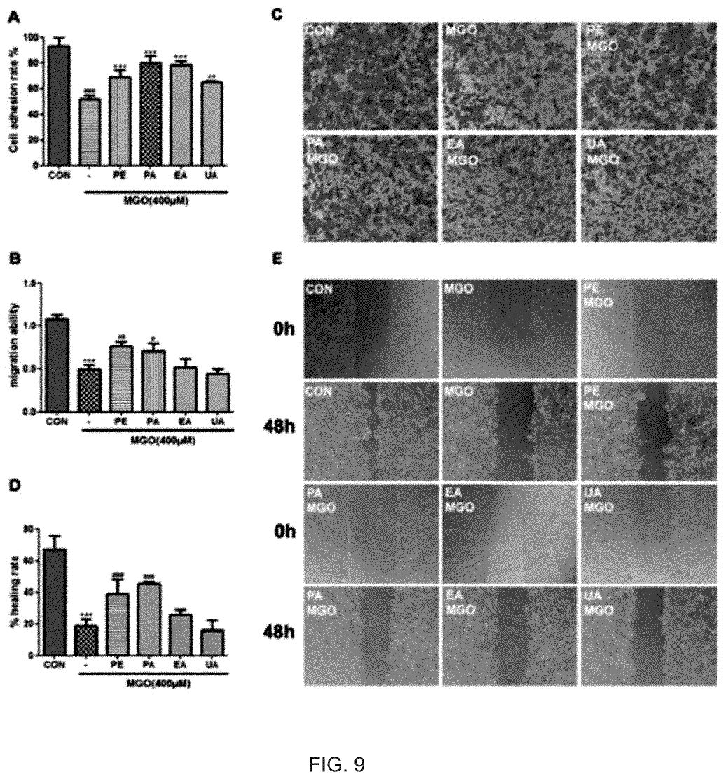

Adhesion assay was carried following previously published protocol.³ HaCaT cells were seeded at about 50 000 cells/ml to yield 50-60% confluency in a standard 6-well plate. Cells were pre-treated with PE 50 μg/mL, PA 50 μM, EA 50 μM, and UA 50 μM, respectively for 2 hours, then washed with PBS twice. After that cells were incubated with 400 μM MGO for 24 hours. After the treatments, the cells were digested with trypsin and centrifuged at 300 g for 5 min. The harvested cells were inoculated on 2 separated 96-well plates, with 12 wells in each group. CTG 2.0 was added six wells of each group to measure the total cells. The cells in the other six wells were cultured for a further 10 h. The medium was removed and the cells were washed with PBS twice to remove the unattached cells, and then the same CTG 2.0 solution was added as above to measure the adhesive cells. The adhesion rate=(the adhesive cells/the total cell)×100%. FIG. 9A and FIG. 9C, show the promotive effects of PE and pomegranate phenolics on cell growth.

Transwell Assay

Cell migration ability was measured by transwell assay. HaCaT cells in 6-well plates were cultured up to about 80-90% confluence. Then cells were pretreated with PE 50 μg/mL, PA 50 μM, EA 50 μM, and UA 50 μM, respectively for 2 hours, then washed with PBS twice. After that, cells were exposed to 400 μM of MGO for 24 h. Then, cells were digested with trypsin and centrifuged at 300 g for 5 min. Then, 2.5×10⁵ cell were suspended in 1 ml serum free DMED. 100 μl of serum free DMEM were added into the upper chamber and incubated for 5 minutes, then 600 μl DMEM containing 5% FBS were added to the 24 well plate (lower chamber). After that 300 μl of cell suspension were added into the upper chamber. After 12 h incubation, cells in upper chamber were washed with PBS and fixed with 75% ethanol for 15 minutes. Then cells in upper chamber were stained with Crystal violet for 10 minutes and washed for 5 times. Cells inside the upper chamber were removed by swab. Then, pictures were taken with EVOS Cell Imaging System (Invitrogen, Waltham, Mass., USA). After that stained cells were incubated in 75% ethanol for 15 minutes for decoloration. Then, optical density of the decoloration solution in 570 nm was calculated via Spectra Max M2 spectrometer (Molecular Devices, Sunnyvale, Calif., USA). FIG. 9B shows the promotive effects of pomegranate compounds, PE, PA, EA, and UA on the cell growth.

Wound Healing Model

Wound healing model was constructed with an in vitro scratch healing assay performed as described previously with certain modifications.⁴ HaCaT cells in 6-well plates were cultured up to about 80-90% confluence. Then cells were pretreated with PE 50 μg/mL, PA 50 μM, EA 50 μM, and UA 50 μM, respectively for 2 hours. After the treatments, a narrow wound-like gap in the cell monolayer was created with a 1000 ml pipette tip in each well. The shedding cells were washed off with PBS and then the images were captured with EVOS Cell Imaging System (Invitrogen, Waltham, Mass., USA). The cells were further exposed to 400 μM MGO for 48 h and the cell images were captured with EVOS Cell Imaging System (Invitrogen, Waltham, Mass., USA). Gap area was analyzed in image J, a software quantifying visualization image data, and the wound healing rate was calculated as follows=[Gap Area(48 h)−Gap Area(0 h)]/Gap Area(0 h)×100%. As shown in FIG. 9D and FIG. 9E, pomegranate extract and PA can promote the wound healing process in the presence of MGO.

Each publication, patent, and patent publication cited in this disclosure is incorporated in reference herein in its entirety. The present invention is not intended to be limited to the foregoing examples, but encompasses all such modifications and variations as come within the scope of the appended claims.

### REFERENCES

1. Yuan T, Ma H, Liu W, et al. Pomegranate's Neuroprotective effects against Alzheimer's disease are mediated by Urolithins, its Ellagitannin-Gut microbial derived metabolites. ACS Chem Neurosci. 2015. doi:10.1021/acschemneuro.5b00260
2. Tang Q, Cai A, Bian K, et al. Characterization of byproducts from chemical syntheses of oligonucleotides containing 1-Methyladenine and 3-Methylcytosine. ACS omega. 2(11):8205-8212.
3. Huang C-Y, Wu T-C, Hong Y-H, Hsieh S-L, Guo H-R, Huang R-H.

Enhancement of Cell Adhesion, Cell Growth, Wound Healing, and Oxidative Protection by Gelatins Extracted from Extrusion-Pretreated Tilapia (Oreochromis sp.) Fish Scale. Molecules. 2018; 23(10):2406.
4. Horikoshi Y, Kamizaki K, Hanaki T, et al. α-Tocopherol promotes HaCaT keratinocyte wound repair through the regulation of polarity proteins leading to the polarized cell migration. BioFactors. 2018; 44(2): 180-191.

We claim:

1. A method of reducing or inhibiting DNA damage in a cell, the method comprising administering an effective amount of a pomegranate polyphenol composition comprising at least a pomegranate polyphenol to reduce or inhibit DNA damage in the cell.

2. The method of claim 1, wherein the pomegranate polyphenol composition comprises a punicalagin-enriched extract from pomegranate.

3. The method of claim 1, wherein the pomegranate polyphenol composition comprises punicalagin, ellagic acid, urolithin A, urolithin B, urolithin C, urolithin D, urolithin E, urolithin M, gallic acid, pomegranate extract, or a combination thereof.

4. The method of claim 3, wherein the pomegranate polyphenol composition comprises a combination of punicalagin and ellagic acid.

5. The method of claim 4, wherein the combination of punicalagin and ellagic acid is from 25% to 40% punicalagin and from 2.0 to 3.0% ellagic acid by weight.

6. The method of claim 3, wherein the pomegranate polyphenol composition comprises pomegranate extract from 3 to 95% by weight of the composition.

7. The method of claim 3, wherein the pomegranate polyphenol composition composition comprises a combination of punicalagins, ellagic acid, and urolithins, wherein the combination of punicalagins, ellagic acid, and urolithins is from 10% to 50% PA, from 0.5% to 5% EA, and from 0.5% to 20% urolithins by weight.

8. The method of claim 1, wherein the cell is an in vivo cell within a subject.

9. The method of claim 8, wherein the cell is a skin cell.

10. The method of claim 1, wherein the DNA damage is from MGO binding, and/or MGO induced protein glycation.

11. A method of reducing or preventing MGO binding, and/or MGO induced protein glycation in a subject in need thereof, the method comprising administering an effective amount of a pomegranate polyphenol composition comprising one or more pomegranate polyphenol to reduce or prevent MGO binding and/or MGO induced protein glycation in at least one cell of the subject.

12. The method of claim 11, wherein the pomegranate polyphenol composition comprises a punicalagin-enriched extract from pomegranate.

13. The method of claim 11, wherein the pomegranate polyphenol composition comprises punicalagin, ellagic acid, urolithin A, urolithin B, urolithin C, urolithin D, urolithin E, urolithin M, gallic acid, pomegranate extract, or a combination thereof.

14. The method of claim 13, wherein the pomegranate polyphenol composition comprises a combination of punicalagin and ellagic acid, wherein the combination of punicalagin and ellagic acid is from 25% to 40% punicalagin and from 2.0 to 3.0% ellagic acid by weight.

15. The method of claim 13, wherein the pomegranate polyphenol composition composition comprises a combination of punicalagins, ellagic acid, and urolithins, wherein the combination of punicalagins, ellagic acid, and urolithins is from 10% to 50% PA, from 0.5% to 5% EA, and from 0.5% to 20% urolithins by weight.

16. The method of claim 13, wherein the pomegranate polyphenol composition comprises pomegranate extract, and wherein the pomegranate extract is from 3% to 95% by weight of the composition.

17. The method of claim 11, wherein the subject is a human.

18. The method of claim 17, wherein the subject has an age-related disease.

19. The method of claim 17, wherein the subject has a skin-related disease.

20. A method of treating a skin damage disease caused by DNA damage, the method comprising administering an effective amount of a pomegranate polyphenol composition comprising at least a pomegranate polyphenol to reduce or inhibit the skin damage.
